(19) United States
(12) Patent Application Publication
(10) Pub. No.: US 2010/0286785 A1
(43) Pub. Date: Nov. 11, 2010

(54) METHOD AND APPARATUS FOR SPINAL INTERBODY FUSION

(76) Inventor: Danny Wayne Grayson, Clemmons, NC (US)

Correspondence Address: Mark J. Pandiscio;Pandiscio & Pandiscio, P.C., 470 Totten Pond Road, Waltham, MA, 02451-1914, US

(21) Appl. No.: 12/772,778

(22) Filed: May 3, 2010

Related U.S. Application Data
(60) Provisional application No. 61/174,975, filed on May 1, 2009.

(51) Int. Cl. A61F 2/44

(52) U.S. Cl. 623/17.16

(57) ABSTRACT

A spinal fusion implant comprising:
a substantially rectangular body having a distal end and a proximal end, and an upper surface and a lower surface; and
an upper stabilizer extending upwardly from the upper surface of the substantially rectangular body, and a lower stabilizer extending downwardly from the lower surface of the substantially rectangular body;
wherein (i) the distance between the distal end of the substantially rectangular body and the proximal end of the substantially rectangular body is long enough to span the distance between opposing cortical portions of a vertebral body and short enough to not extend substantially beyond the vertebral body, and (ii) the distance between the upper surface of the substantially rectangular body and the lower surface of the substantially rectangular body is substantially the same as the gap between opposing vertebral bodies in a spinal joint.

### REFERENCE TO PENDING PRIOR PATENT APPLICATION

This patent application claims benefit of pending prior U.S. Provisional Patent Application Ser. No. 61/174,975, filed May 1, 2009 by Danny Wayne Grayson for METHOD AND APPARATUS FOR SPINAL INTERBODY FUSION (Attorney's Docket No. VG-3 PROV), which patent application is hereby incorporated herein by reference.

### FIELD OF THE INVENTION

This invention relates to surgical methods and apparatus in general, and more particularly to surgical methods and apparatus for fusing spinal vertebral bodies.

### BACKGROUND OF THE INVENTION

Disc herniation is a condition in which a spinal disc bulges from between two vertebral bodies and impinges on adjacent nerves, thereby causing pain. In some cases, non-operative procedures (including bed rest, medication, lifestyle modifications, exercise, physical therapy, chiropractic care and steroid injections) may be satisfactory treatment. However, in other cases, surgical intervention may be necessary. In cases where surgical intervention is prescribed, spinal vertebral body fusion may be desirable.

More particularly, the current standard of care for surgically treating disc herniation in patients who have chronic pain and who have (or who are likely to develop) associated spinal instability is spinal fixation. Spinal fixation procedures are intended to relieve the impingement on the nerves by removing the portion of the disc and/or bone responsible for compressing the neural structures and destabilizing the spine. The excised disc or bone is replaced with one or more intervertebral implants, or spacers, placed between the adjacent vertebral bodies. These implants stabilize the adjacent vertebral bodies relative to one another so that the two vertebral bodies can fuse together.

### SUMMARY OF THE INVENTION

The present invention provides a novel method and apparatus for effecting spinal vertebral body fusion. More particularly, the present invention comprises the provision and use of a novel spinal fusion implant for disposition between adjacent vertebral bodies, whereby to immobilize the affected segment and facilitate fusion between the opposing vertebral bodies.

In one preferred form of the invention, there is provided a spinal fusion implant comprising:

a substantially rectangular body having a distal end and a proximal end, and an upper surface and a lower surface; and

an upper stabilizer extending upwardly from the upper surface of the substantially rectangular body, and a lower stabilizer extending downwardly from the lower surface of the substantially rectangular body;

wherein (i) the distance between the distal end of the substantially rectangular body and the proximal end of the substantially rectangular body is long enough to span the distance between opposing cortical portions of a vertebral body and short enough to not extend substantially beyond the vertebral body, and (ii) the distance between the upper surface of the substantially rectangular body and the lower surface of the substantially rectangular body is substantially the same as the gap between opposing vertebral bodies in a spinal joint.

In another preferred form of the invention, there is provided a method for fusing a spinal joint, the method comprising the steps of:

providing a spinal fusion implant comprising:
a substantially rectangular body having a distal end and a proximal end, and an upper surface and a lower surface; and
an upper stabilizer extending upwardly from the upper surface of the substantially rectangular body, and a lower stabilizer extending downwardly from the lower surface of the substantially rectangular body;
wherein (i) the distance between the distal end of the substantially rectangular body and the proximal end of the substantially rectangular body is long enough to span the distance between opposing cortical portions of a vertebral body and short enough to not extend substantially beyond the vertebral body, and (ii) the distance between the upper surface of the substantially rectangular body and the lower surface of the substantially rectangular body is substantially the same as the gap between opposing vertebral bodies in a spinal joint;

deploying the spinal fusion implant in the spinal joint so that the substantially rectangular body is disposed between the opposing vertebral bodies of the spinal joint, with the upper stabilizer projecting into the upper vertebral body and the lower stabilizer projecting into the lower vertebral body; and

maintaining the spinal fusion implant in this position while fusion occurs.

In another preferred form of the invention, there is provided a fusion implant comprising:

a substantially rectangular body having a distal end and a proximal end, and a first surface and a second surface, the first surface and the second surface facing in substantially opposite directions; and

a first stabilizer extending away from the first surface of the substantially rectangular body, and a second stabilizer extending away from the second surface of the substantially rectangular body;

wherein (i) the distance between the distal end of the substantially rectangular body and the proximal end of the substantially rectangular body is long enough to span the distance between opposing cortical portions of a bone and short enough to not extend substantially beyond the bone, and (ii) the distance between the first surface of the substantially rectangular body and the second surface of the substantially rectangular body is substantially the same as the gap between opposing bones in a joint.

In another preferred form of the invention, there is provided a method for fusing a joint, the method comprising the steps of:

providing a fusion implant comprising:
a substantially rectangular body having a distal end and a proximal end, and a first surface and a second surface, the first surface and the second surface facing in substantially opposite directions; and
a first stabilizer extending away from the first surface of the substantially rectangular body, and a second stabilizer extending away from the second surface of the substantially rectangular body;
wherein (i) the distance between the distal end of the substantially rectangular body and the proximal end of the substantially rectangular body is long enough to span the distance between opposing cortical portions of a bone and short enough to not extend substantially beyond the bone, and (ii) the distance between the first surface of the substantially rectangular body and the second surface of the substantially rectangular body is substantially the same as the gap between opposing bones in a joint;

deploying the fusion implant in the joint so that the substantially rectangular body is disposed between the opposing bones of the joint, with the upper stabilizer projecting into one bone of the joint and the lower stabilizer projecting into bone of the joint; and

maintaining the fusion implant in this position while fusion occurs.

In another preferred form of the invention, there is provided a fusion implant comprising:

a body having a distal end and a proximal end, and a first surface and a second surface, the first surface and the second surface facing in different directions; and

at least one stabilizer extending away from the body;

wherein (i) the distance between the distal end of the body and the proximal end of the body is long enough to span the distance between opposing cortical portions of a bone and short enough to not extend substantially beyond the bone, and (ii) the distance between the first surface of the body and the second surface of the body is substantially the same as the gap between opposing bones in a joint.

In another preferred form of the invention, there is provided a method for fusing a joint, the method comprising the steps of:

providing a fusion implant comprising:
a body having a distal end and a proximal end, and a first surface and a second surface, the first surface and the second surface facing in different directions; and
at least one stabilizer extending away from the body;
wherein (i) the distance between the distal end of the body and the proximal end of the body is long enough to span the distance between opposing cortical portions of a bone and short enough to not extend substantially beyond the bone, and (ii) the distance between the first surface of the body and the second surface of the body is substantially the same as the gap between opposing bones in a joint;

deploying the fusion implant in the joint so that the body is disposed between the opposing bones of the joint, with the at least one stabilizer projecting into one bone of the joint; and

maintaining the fusion implant in this position while fusion occurs.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of a human spine;

FIGS. 2-5 are schematic views illustrating a first embodiment of a spinal fusion implant formed in accordance with the present invention;

FIG. 6 is a schematic view illustrating the spinal fusion implant of FIGS. 2-5 installed in a disc space;

FIGS. 7-9 are schematic views illustrating different sizes of the spinal fusion implant installed in a disc space;

FIG. 10 is a schematic view like that of FIG. 6, but showing one or more plates overlying the spinal fusion implant;

FIGS. 11-14 are schematic views illustrating a second embodiment of a spinal fusion implant formed in accordance with the present invention;

FIGS. 15 and 16 are schematic views illustrating a third embodiment of a spinal fusion implant formed in accordance with the present invention;

FIGS. 17 and 18 are schematic views illustrating a fourth embodiment of a spinal fusion implant formed in accordance with the present invention;

FIGS. 19-21 are schematic views illustrating a fifth embodiment of a spinal fusion implant formed in accordance with the present invention;

FIGS. 22 and 23 are schematic views illustrating a sixth embodiment of a spinal fusion implant formed in accordance with the present invention;

FIGS. 24-26 are schematic views illustrating a seventh embodiment of a spinal fusion implant formed in accordance with the present invention;

FIGS. 27 and 28 are schematic views illustrating an eighth embodiment of a spinal fusion implant formed in accordance with the present invention;

FIGS. 29 and 30 are schematic views illustrating a ninth embodiment of a spinal fusion implant formed in accordance with the present invention;

FIGS. 31-33 are schematic views illustrating a tenth embodiment of a spinal fusion implant formed in accordance with the present invention;

FIGS. 34-36 are schematic views illustrating an eleventh embodiment of a spinal fusion implant formed in accordance with the present invention;

FIGS. 37 and 38 are schematic views illustrating a twelfth embodiment of a spinal fusion implant formed in accordance with the present invention;

FIGS. 39-42 are schematic views illustrating a thirteenth embodiment of a spinal fusion implant formed in accordance with the present invention;

FIGS. 43-45 are schematic views illustrating a fourteenth embodiment of a spinal fusion implant formed in accordance with the present invention;

FIGS. 46-49 are schematic views illustrating a fifteenth embodiment of a spinal fusion implant formed in accordance with the present invention; and

FIGS. 50-52 are schematic views illustrating a sixteenth embodiment of a spinal fusion implant formed in accordance with the present invention.

### DETAILED DESCRIPTION OF THE INVENTION

### The Spinal Fusion Implant in General

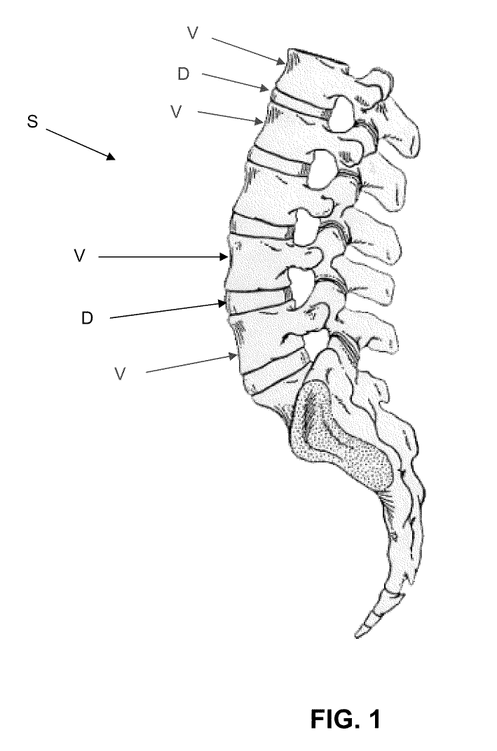

Looking first at FIG. 1, there is shown a typical human spine S. Spine S generally comprises a plurality of vertebral bodies V separated by discs D. As noted above, in some circumstances, the spine may deteriorate so that adjacent vertebral bodies must be fused together. The present invention comprises the provision and use of a novel spinal fusion implant for disposition between the vertebral bodies, whereby to immobilize the affected segment of the spine and facilitate fusion between the opposing vertebral bodies.

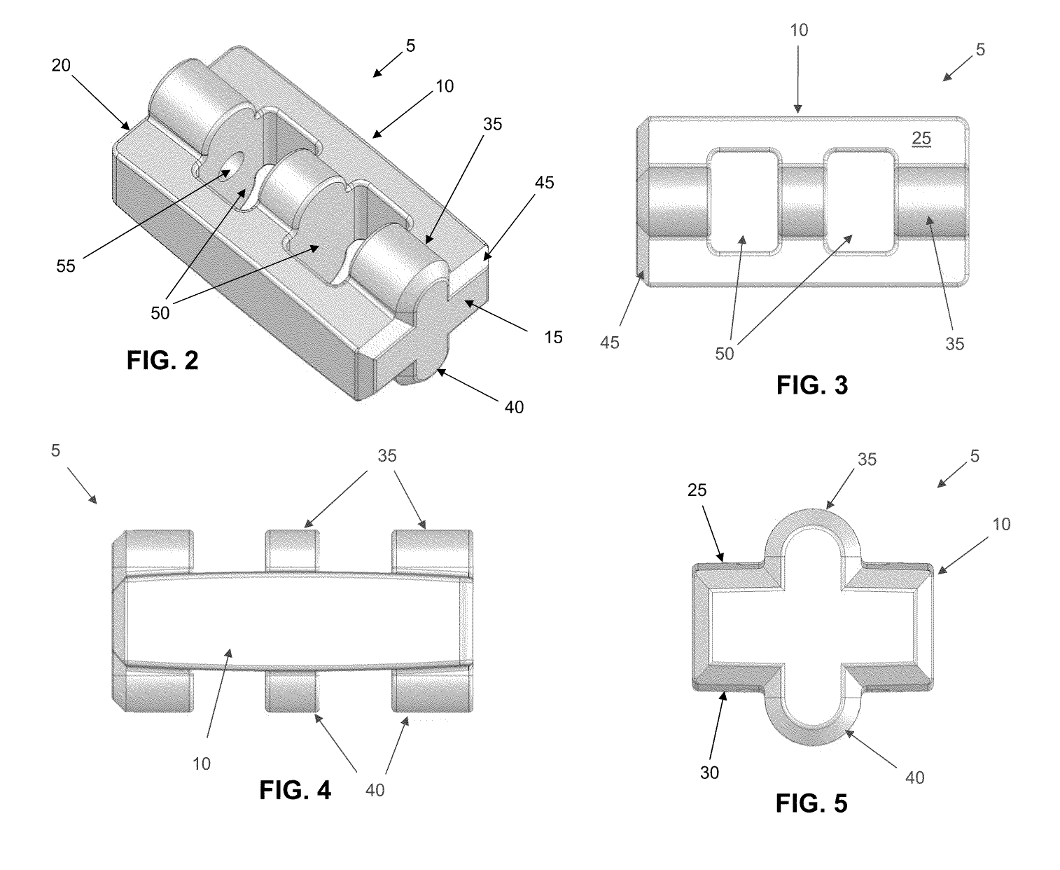

More particularly, and looking now at FIGS. 2-5, there is shown a spinal fusion implant 5 formed in accordance with the present invention. Spinal fusion implant 5 generally comprises a substantially rectangular body 10 having a distal end 15 and a proximal end 20, and an upper surface 25 and a lower surface 30. In one preferred form of the present invention, and as shown in FIGS. 2, 4 and 5, upper surface 25 and lower surface 30 extend substantially parallel to one another.

Spinal fusion implant 5 also comprises an upper stabilizer 35 protruding upwardly from upper surface 25, and a lower stabilizer 40 protruding downwardly from lower surface 30. In one preferred form of the present invention, upper stabilizer 35 and lower stabilizer 40 extend along substantially the entire length of substantially rectangular body 10 (FIG. 2), and have a generally arcuate (FIG. 5) configuration (i.e., upper stabilizer 35 and lower stabilizer 40 have generally semi-circular cross-sections). In one preferred form of the invention, upper stabilizer 35 and lower stabilizer 40 protrude upwardly and downwardly, respectively, a sufficient distance that the stabilizers will project into the cancellous bone of an adjacent vertebral body, as will hereinafter be discussed.

And in one preferred form of the present invention, and as shown in FIGS. 2-5, spinal fusion implant 5 includes tapered surfaces 45 at its distal end 15 and one or more vertical holes 50 extending into substantially rectangular body 10 and/or upper stabilizer 35 and lower stabilizer 40. In one preferred form of the invention, one or more vertical holes 50 extend through the complete height of spinal fusion implant 5. Preferably, and as seen in FIG. 2, spinal fusion implant 5 also includes a longitudinally-extending opening 55 formed in at least its proximal end for receiving an inserter, whereby spinal fusion implant 5 may be manipulated for implantation in the body as will hereinafter be discussed in further detail.

### Use of the Spinal Fusion Implant

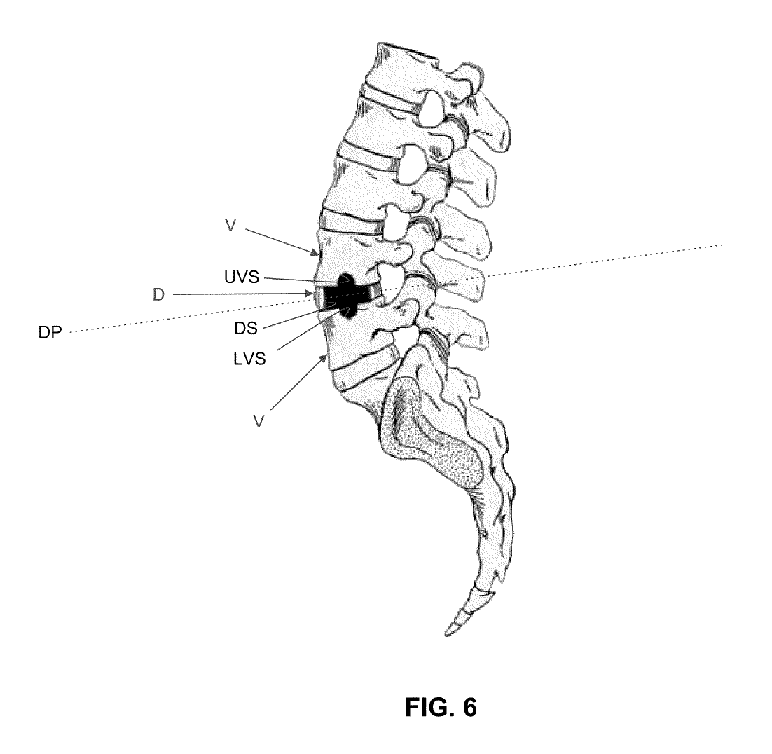

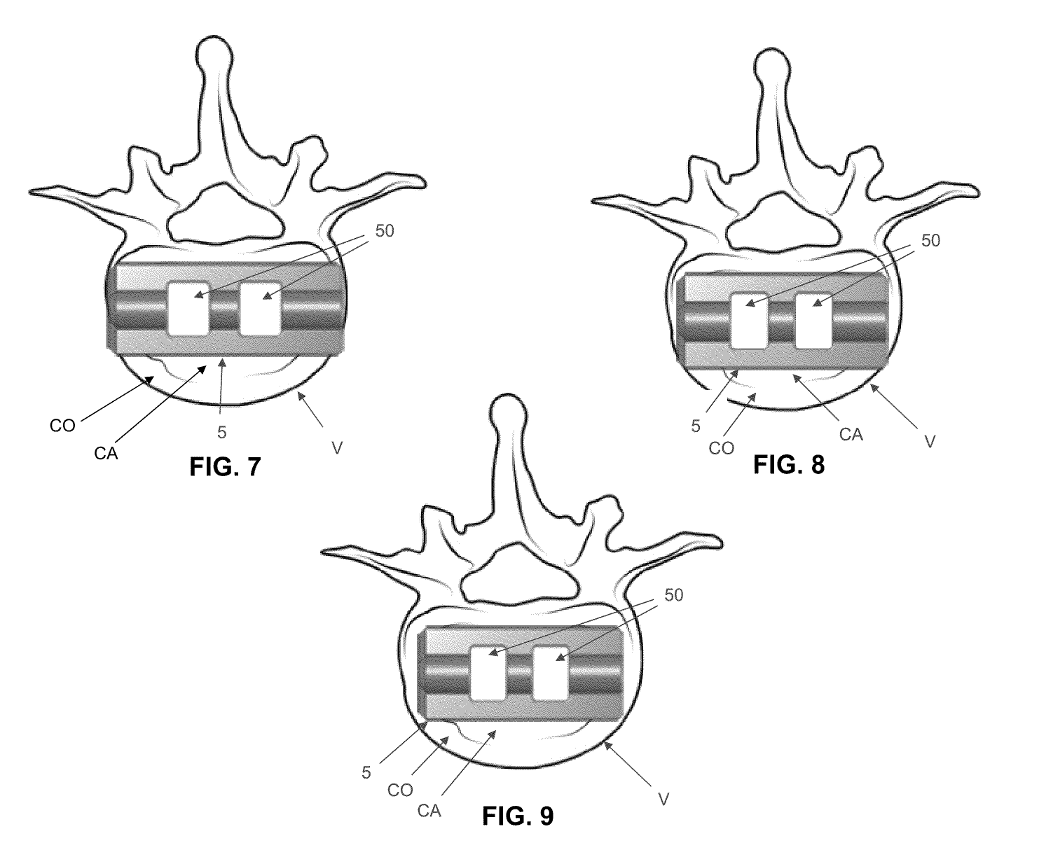

As seen in FIGS. 6-9, spinal fusion implant 5 is intended to be disposed between, and extend into, two adjacent vertebral bodies V so as to stabilize those two vertebral bodies relative to one another and permit fusion of the same. To this end, and looking now at FIG. 6, the spine is prepared by removing some or all of the disc which resides in the space which is to be occupied by spinal fusion implant 5, e.g., a disc space DS is prepared with a rongeur or other surgical instrument. In addition, portions of the upper and lower vertebral bodies are removed so as to prepare seats for upper stabilizer 35 and lower stabilizer 40, e.g., an upper vertebral body space UVS is prepared in the upper vertebral body with a drill (or other surgical instrument) and a lower vertebral body space LVS is prepared in the lower vertebral body with a drill (or other surgical instrument). In one preferred form of the invention, upper vertebral body space UVS extends into the cancellous interior of the upper vertebral body and lower vertebral body space LVS extends into the cancellous interior of the lower vertebral body. Then, as seen in FIGS. 6-9, spinal fusion implant 5 is advanced horizontally, distal end first, into the gap between the upper and lower vertebral bodies so that substantially rectangular body 10 is disposed in disc space DS, with upper surface 25 engaging the lower endplate of the upper vertebral body and lower surface 30 engaging the upper endplate of the lower vertebral body, and with upper stabilizer 35 seated in upper vertebral body space UVS (and preferably engaging the cancellous bone of the upper vertebral body) and with lower stabilizer 40 seated in lower vertebral body space LVS (and preferably engaging the cancellous bone of the lower vertebral body). In this respect, it will be appreciated that spinal fusion implant 5 is sized so that the distance between upper surface 25 and lower surface 30 is substantially the same as the height of the disc that the spinal fusion implant is to replace, so that the proper spacing of the vertebral bodies can be maintained. In addition, it will be appreciated that spinal fusion implant 5 is sized so that it can span, in a lateral direction, cancellous portion CA of vertebral body V, with its proximal and distal ends resting on diametrically opposed portions CO of vertebral body V. At the same time, spinal fusion implant 5 is sized so that it can cover, in an anterior-posterior direction, a substantial portion of the endplate of the vertebral body, e.g., preferably at least 45% of the anterior-posterior dimension of the endplate, and more preferably about 60%+ of the anterior-posterior dimension of the endplate. As a result, spinal fusion implant 5 supports the affected segment of the spine and, by virtue of disposition of upper stabilizer 35 in the upper vertebral body and the disposition of lower stabilizer 40 in the lower vertebral body, immobilizes the affected segment of the spine, thereby facilitating fusion between the opposing vertebral bodies. In this respect it will also be appreciated that vertical holes 50 of spinal fusion implant 5 permit the cancellous bone CA of the upper vertebral body and the cancellous bone CA of the lower vertebral body to grow into spinal fusion implant 5, whereby to further facilitate bone fusion. If desired, vertical holes 50 may be filled with a bone growth promoter.

In one preferred form of the present invention, spinal fusion implant 5 is intended to be inserted into a disc space using a lateral or anterior approach. The lateral or anterior approach is generally preferred since it is familiar to spine surgeons, and also minimizes the possibility of damage to the spinal cord during insertion of the spinal fusion implant.

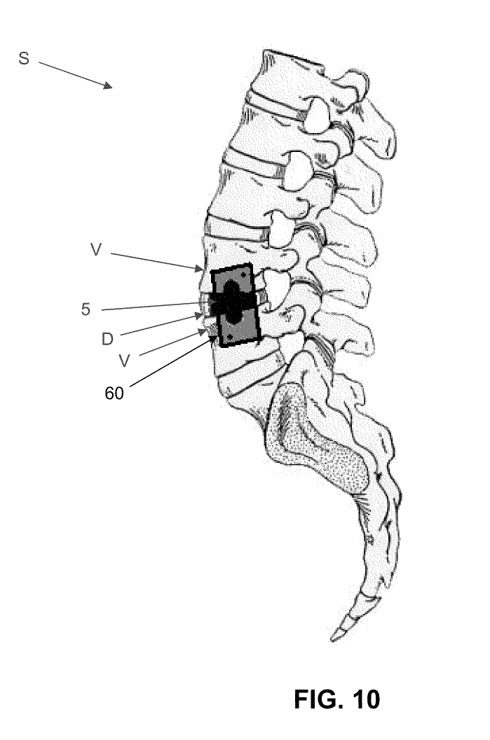

If desired, and looking now at FIG. 10, one or more plates 60 may be applied to the upper and lower vertebral bodies so as to help lock spinal fusion implant 5 into position.

In one preferred manner of use, and looking again at FIG. 6, an instrument is first used to determine the disc plane VP of the disc space which is to receive spinal fusion implant 5. Properly identifying the disc plane of the disc space is generally important, since disc plane VP may be used to identify the proper position for disc space DS and upper vertebral space UVS and lower vertebral space LVS which are created to receive the spinal fusion implant.

In this respect it should be appreciated that at least one of the instruments preferably comprises a directional feature which is used to maintain alignment of the instrumentation with the vertical plane of the intervertebral joint. By way of example but not limitation, a directional cannula may comprise a flat portion and the remaining instruments may comprise a flat portion on an opposite portion of the instrument, so that the instruments may only be inserted through the cannula at 0 degrees and/or 180 degrees.

After the proper position for disc space DS and upper vertebral space UVS and lower vertebral space LVS have been identified, a drill (or reamer, punch, dremel, router, burr, etc.) is preferably used to form the desired cavities in the disc and in the opposing vertebral bodies.

After disc space DS and upper vertebral space UVS and lower vertebral space LVS have been formed in the disc space and the opposing vertebral bodies, respectively, spinal fusion implant 5 is inserted into disc space DS and upper vertebral space UVS and lower vertebral space LVS so that (i) substantially rectangular body 10 spans the gap between the opposing vertebral bodies, with lower surface 30 resting on the upper endplate of the lower vertebral body and upper surface 25 supporting the lower endplate of the upper vertebral body, and (ii) upper stabilizer 35 and lower stabilizer 40 extend into the opposing vertebral bodies in a sort of “tongue-and-groove” configuration, whereby to lock the upper and lower vertebral bodies against lateral and torsional movement, etc. relative to spinal fusion implant 5 and relative to each other. Preferably, spinal fusion implant 5 is slightly oversized relative to disc space DS and upper vertebral space UVS and lower vertebral space LVS so as to create a press fit. Spinal fusion implant 5 provides the stability and strength needed to immobilize the vertebral bodies while fusion occurs. Due to the non-circular cross-section of substantially rectangular body 10 and the disposition of upper stabilizer 35 and lower stabilizer 40 into the opposing vertebral bodies, spinal fusion implant 5 will hold the opposing vertebral bodies stable relative to one another.

It should be appreciated that where the spinal fusion implant 5 is formed out of a sufficiently strong and rigid material, disc space DS and upper vertebral space UVS and lower vertebral space LVS may not need to be pre-formed in the disc and the opposing vertebral bodies. In this case, the spinal fusion implant may be able to be simply tapped into place, in much the same manner that a punch is used.

Thus it will be seen that the present invention provides a new and improved spinal fusion implant for facilitating vertebral body fusion. This new spinal fusion implant is able to withstand greater forces, prohibit motion in all directions and drastically reduce the risk of implant failure. The new spinal fusion implant also eliminates the possibility of slippage during spinal motion, greatly improves vertebral body stability and promotes better inter-vertebral body fusion.

It should be appreciated that the new spinal fusion implant combines two unique “shapes” in one implant (i.e., the shape of substantially rectangular body 10 and the shape of upper stabilizer 35 and lower stabilizer 40) in order to limit motion in a multi-directional joint. More particularly, the shape of substantially rectangular body 10 limits motion in flexion/extension, while the shape of upper stabilizer 35 and lower stabilizer 40 (i.e., the “keels”) rest within the two bony structures and limits lateral bending. This construction eliminates the possibility of eccentric forces inducing motion in the joint.

Furthermore, and significantly, it has been found that while the present invention effectively stabilizes the joint, it still permits the occurrence of “micro-motion” between the opposing vertebral bodies, which research suggests is important for successful bone fusion.

It should be appreciated that the new spinal fusion implant may be manufactured in a wide range of different sizes in order to accommodate any size of disc between the vertebral bodies.

Furthermore, if desired, the upper and lower surfaces 25, 30 of substantially rectangular body 10 may be formed with an inclined (i.e., non-parallel) orientation so as to provide the spinal fusion implant with an overall wedge shape, whereby to provide spinal curvature where desired.

Additionally, the new spinal fusion implant may be constructed out of substantially any biocompatible material which has properties consistent with the present invention including, but not limited to, allograft, autograft, synthetic bone, simulated bone material, biocomposites, ceramics, PEEK, stainless steel and titanium. Thus, the present invention permits the surgeon to select a spinal fusion implant having the appropriate size and composition for a given intervertebral fusion.

### Alternative Constructions

The configuration of spinal fusion implant 5 may be varied without departing from the scope of the present invention.

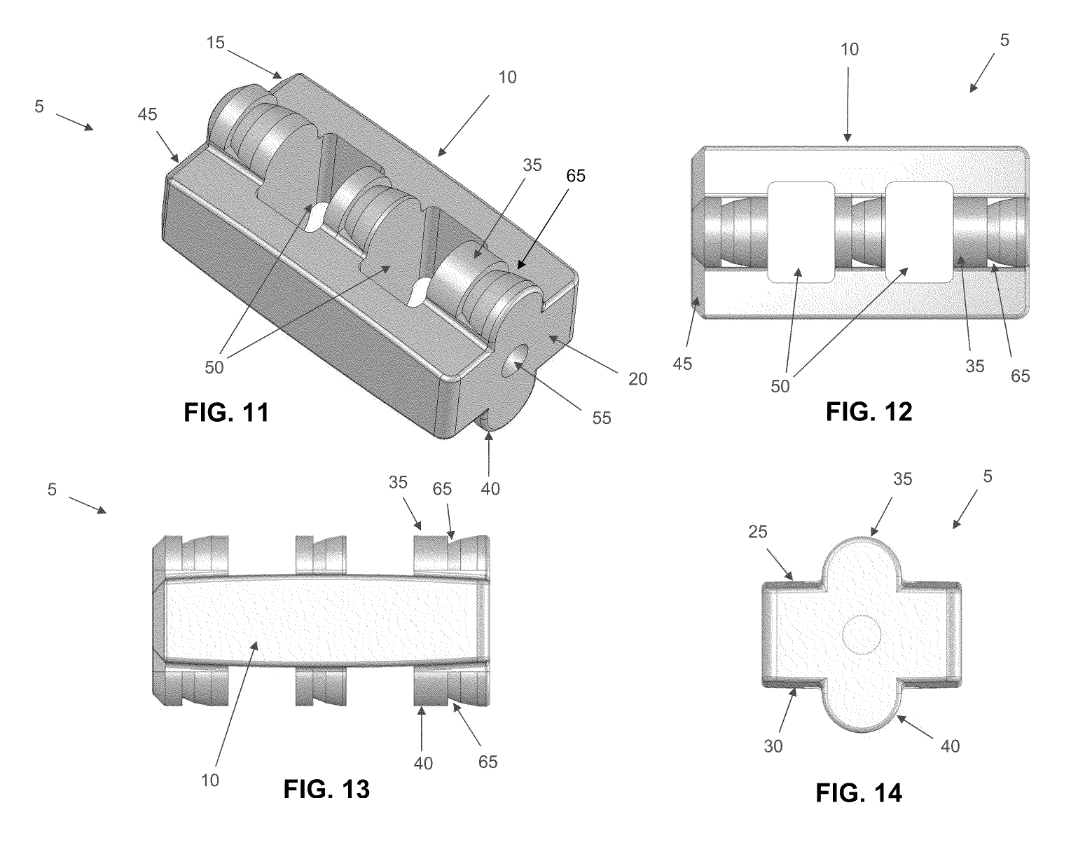

In one configuration, and looking now at FIGS. 11-14, spinal fusion implant 5 may have its upper stabilizer 35 and/or its lower stabilizer 40 sculpted, as shown at 65, so as to provide a frusto-conical configuration. Such a configuration can be advantageous to prevent implant pull-out.

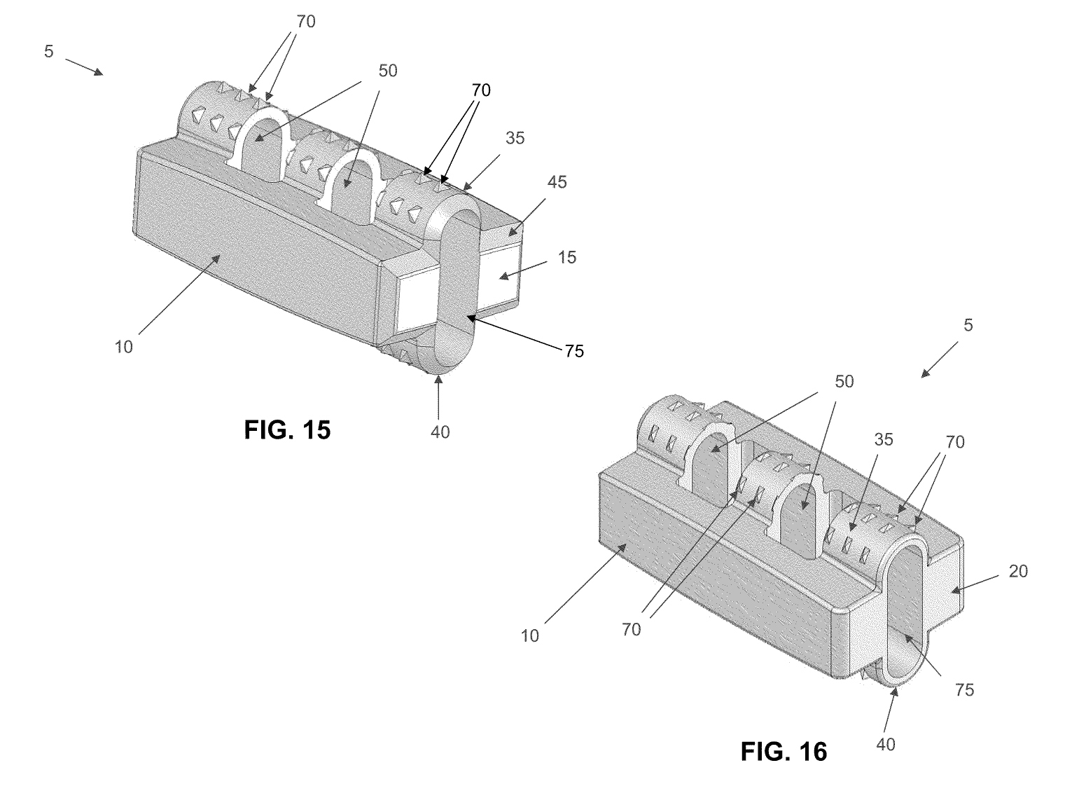

In another configuration, and looking now at FIGS. 15 and 16, sharp barbs 70 may be provided on the outer surface of spinal fusion implant 5 (e.g., on upper stabilizer 35, lower stabilizer 40, substantially rectangular body 10, etc.) so as to further stabilize spinal fusion implant 5 vis-à-vis one or both of the opposing vertebral bodies. Furthermore, if desired, longitudinally-extending opening 55 may be substantially enlarged, e.g., as shown at 75, and extend along the complete length of the spinal fusion implant, so as to render the interior of spinal fusion implant 5 substantially hollow.

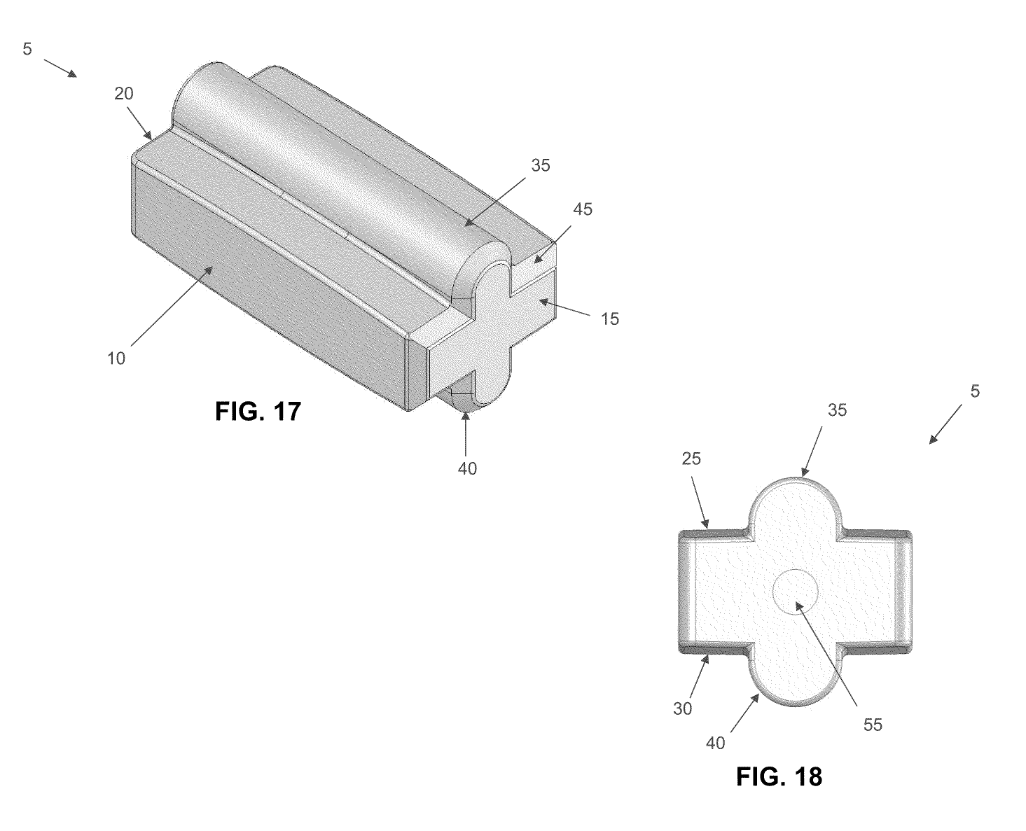

In another configuration, and looking now at FIGS. 17 and 18, spinal fusion implant 5 may be formed substantially solid, with or without longitudinally-extending opening 55.

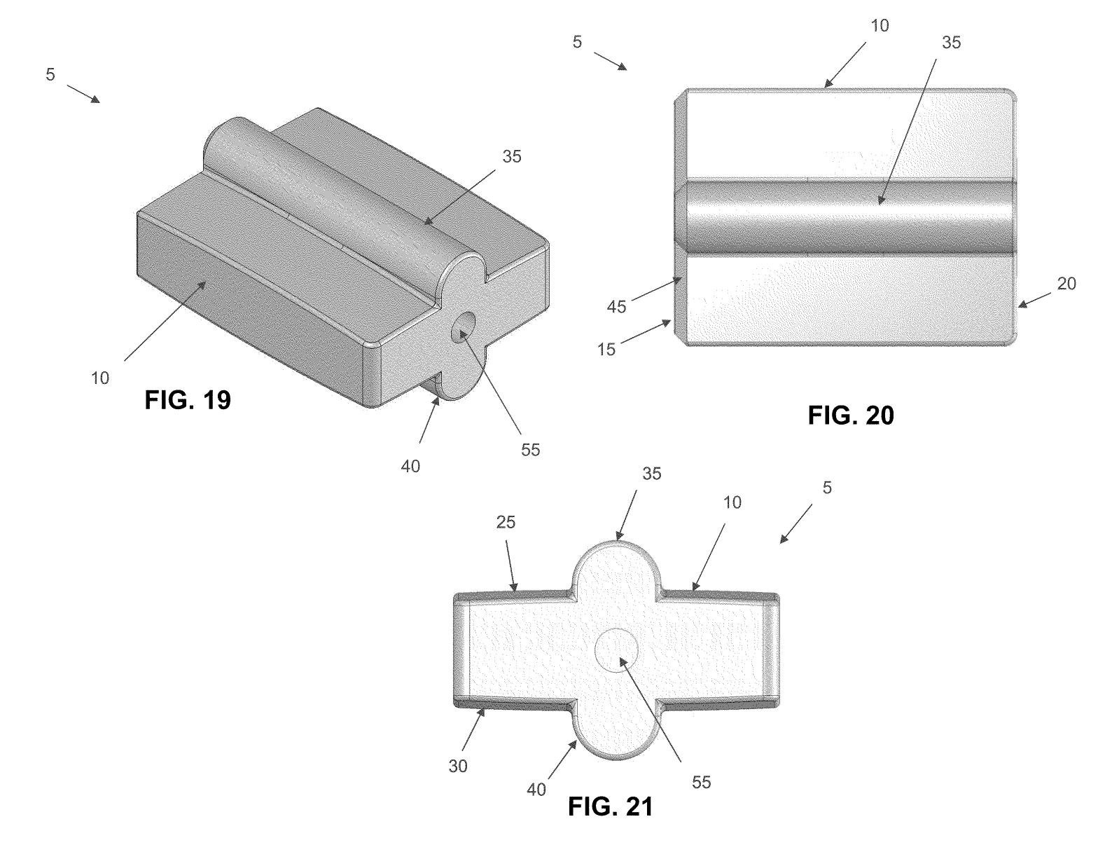

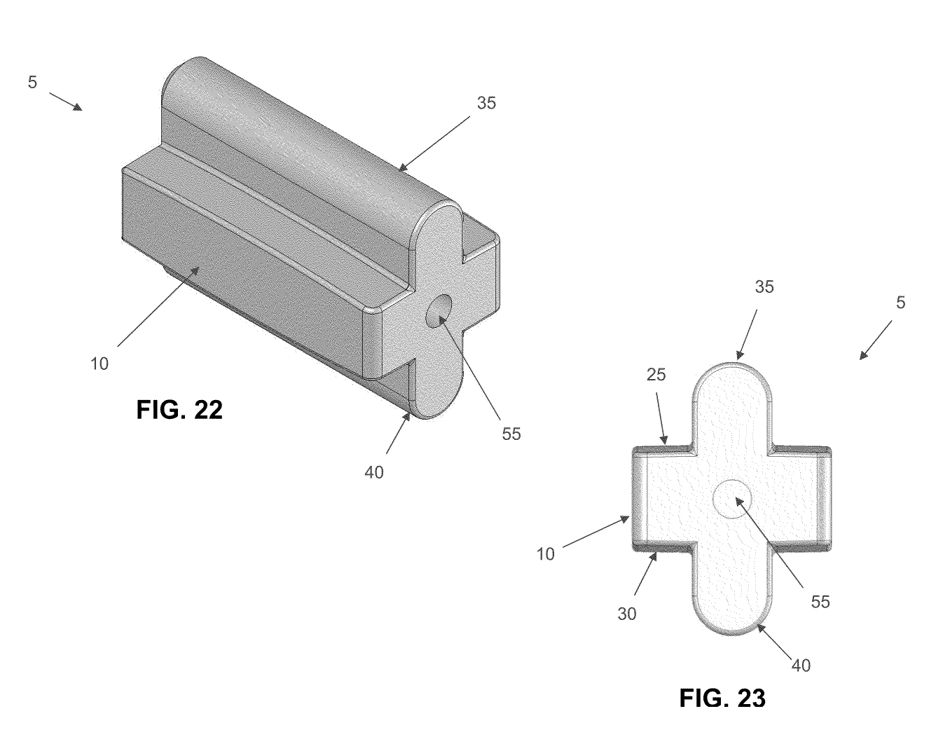

Furthermore, as seen in FIGS. 19-21, and FIGS. 22 and 23, the various proportions of spinal fusion implant 5 may be adjusted.

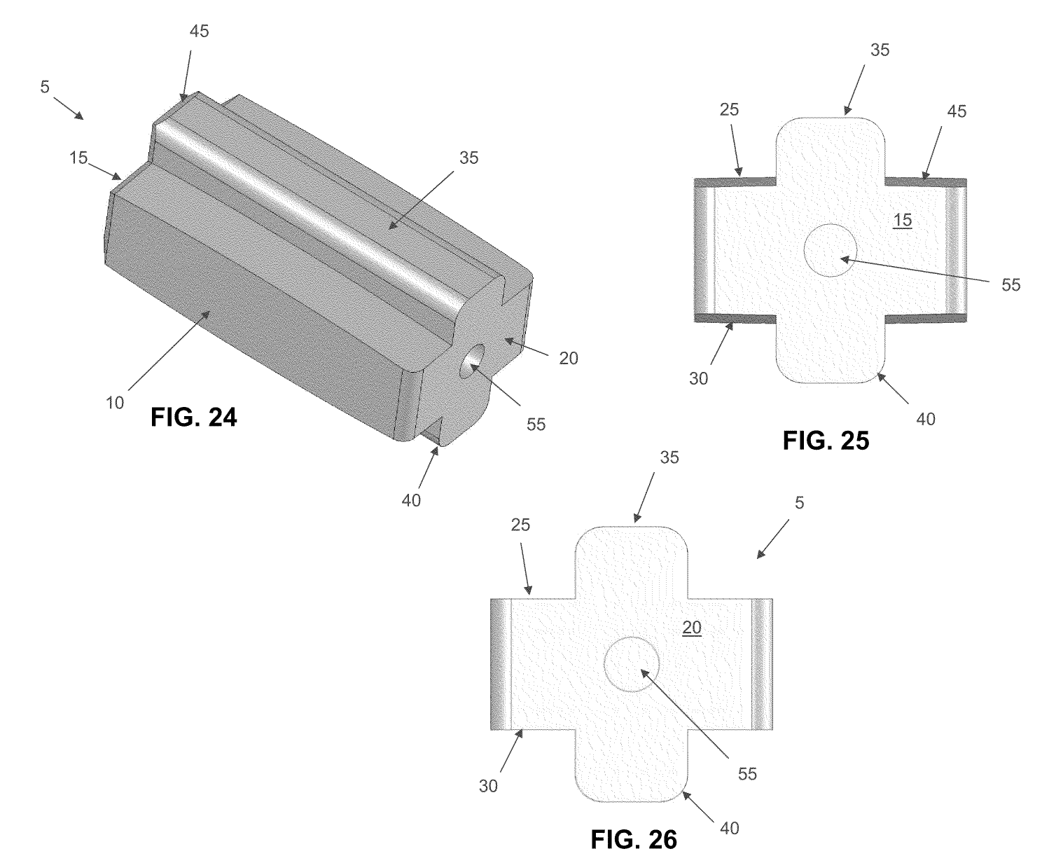

And as seen in FIGS. 24-26, upper stabilizer 35 and/or lower stabilizer 40 may be provided with rounded rectangular configurations, rather than the generally arcuate configuration of FIGS. 2-5, or upper stabilizer 35 and/or lower stabilizer 40 can be provided with other cross-sectional configurations, e.g., a substantially rectangular configuration, a substantially triangular configuration, etc.

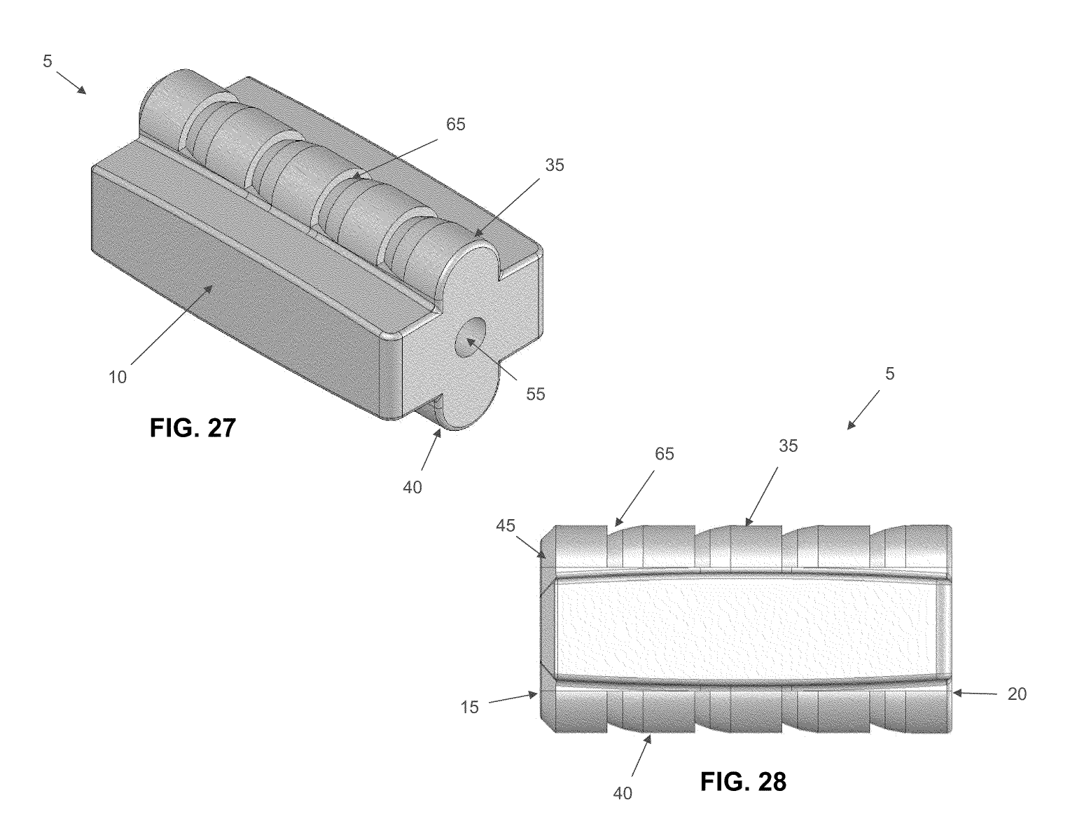

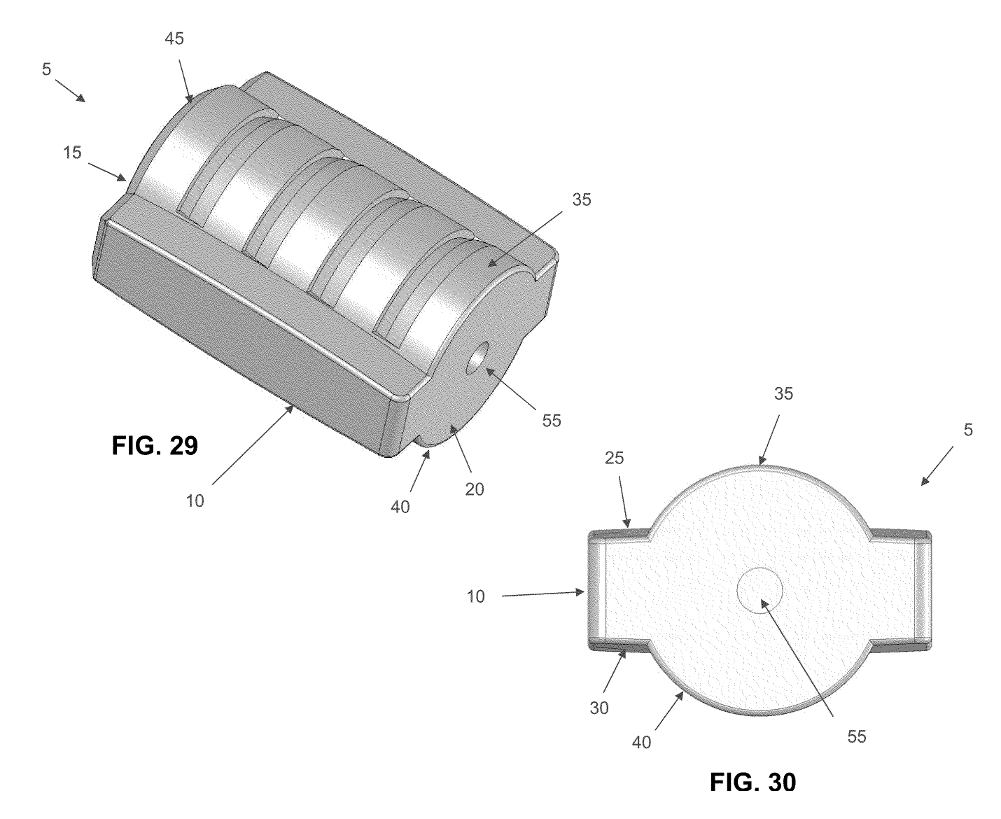

FIGS. 27 and 28, and FIGS. 29 and 30, show how upper stabilizer 35 and lower stabilizer 40 may be provided with a variety of configurations.

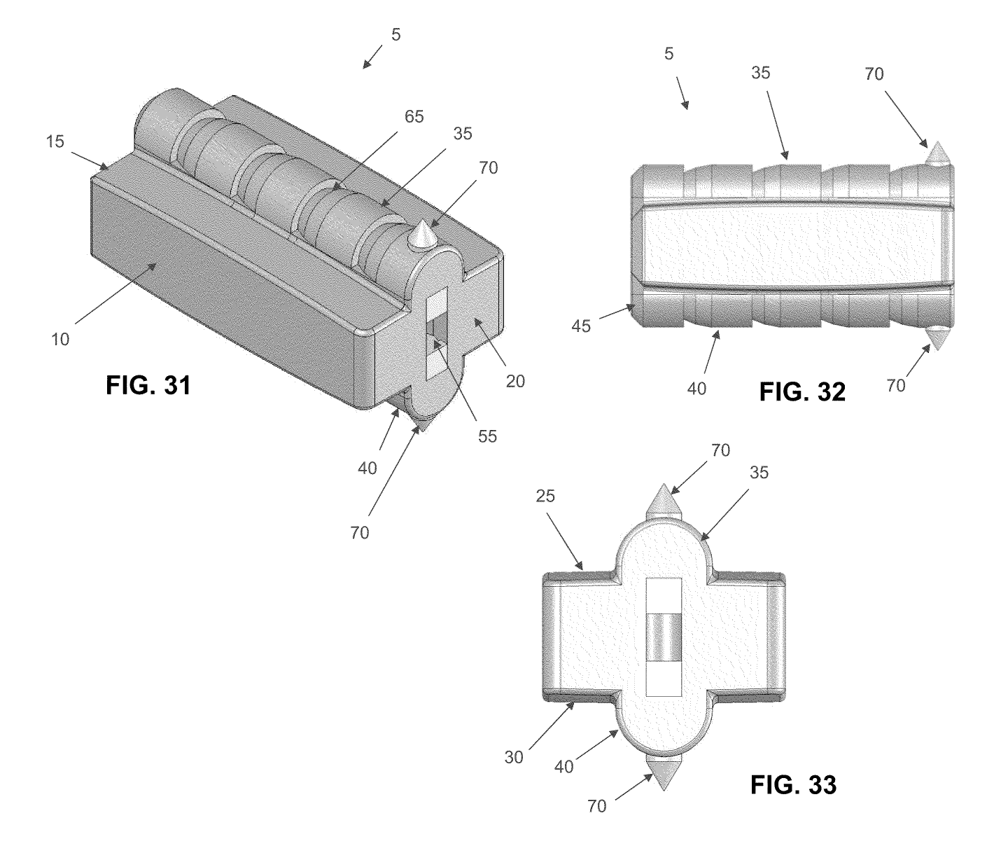

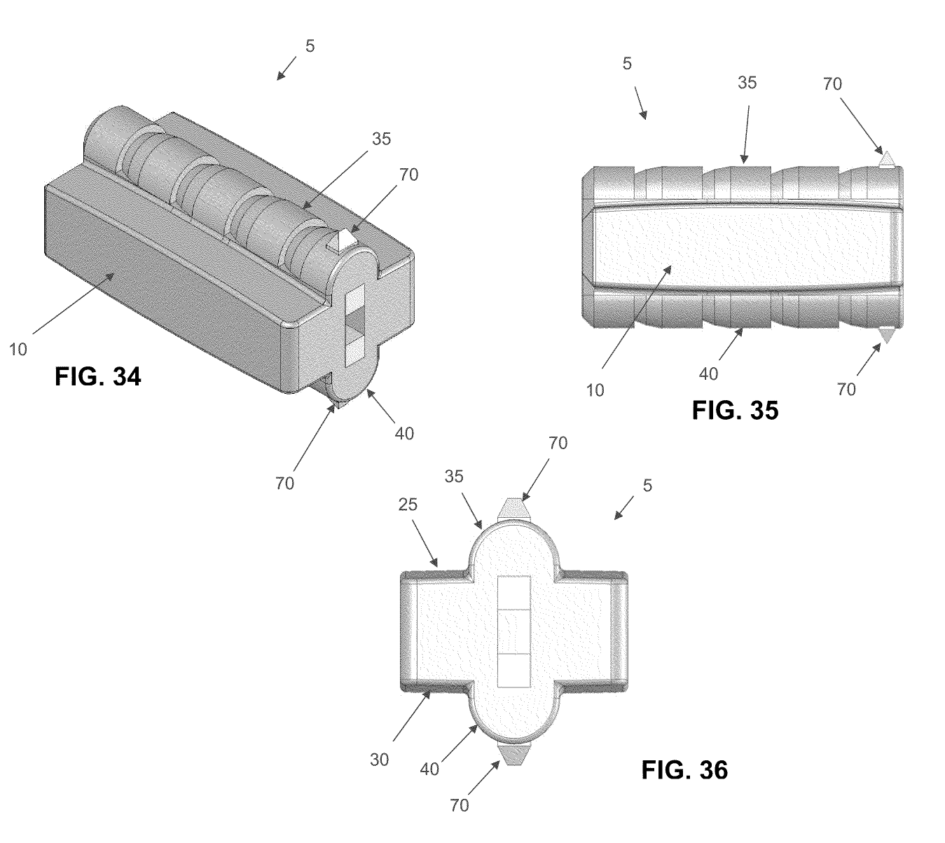

FIGS. 31-33, and FIGS. 34-36, show how a single large barb 70 may be provided on upper stabilizer 35 and/or lower stabilizer 40 so as to stabilize spinal fusion body 5 relative to an adjacent vertebral body, by being pushed into the bony structure, with barb 70 having various configurations.

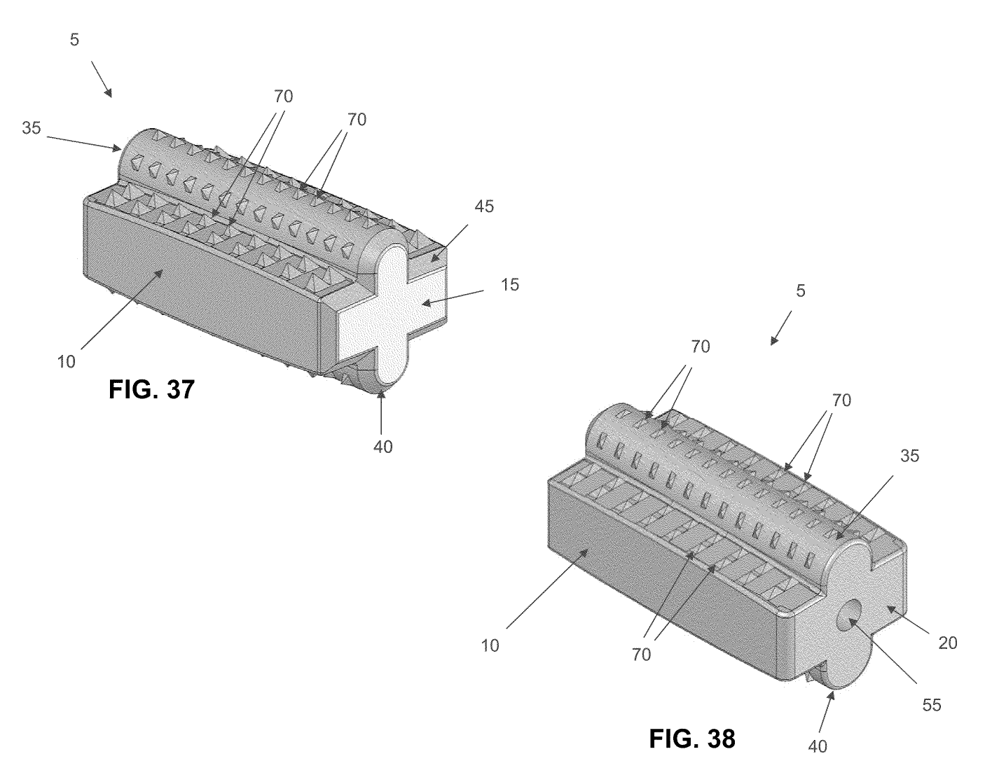

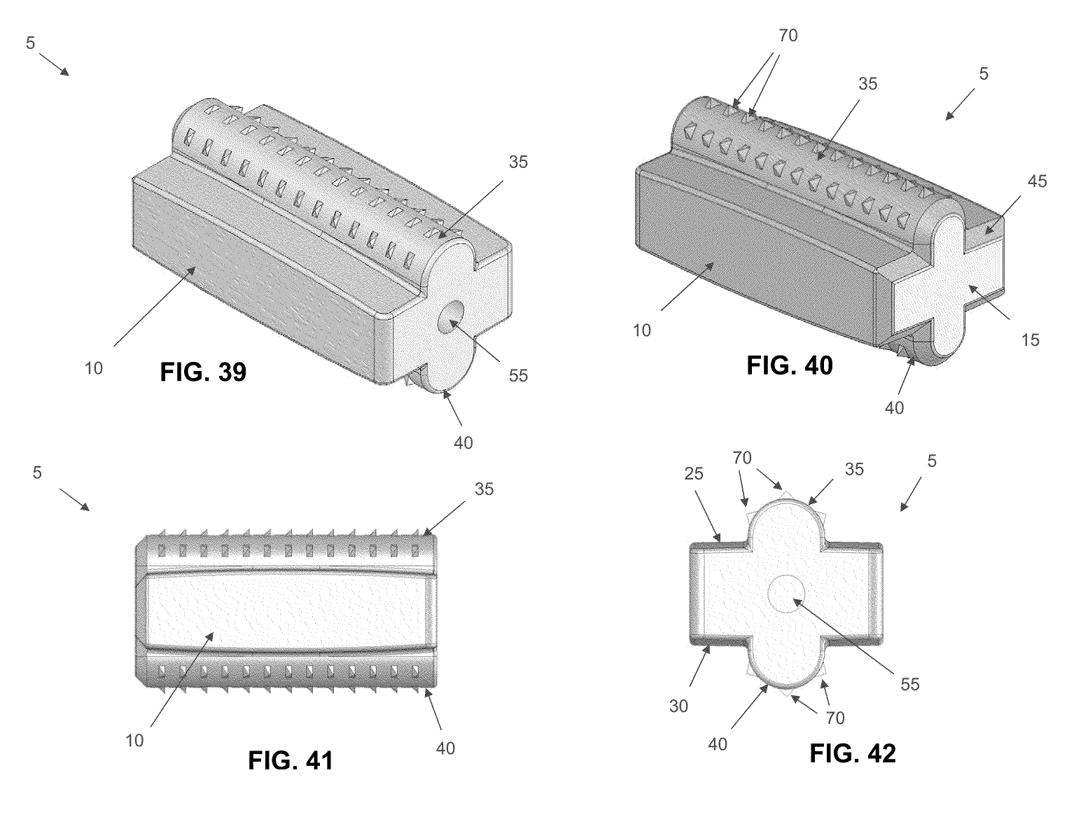

FIGS. 37-38, and FIGS. 39-42, show how barbs 70 may be applied across various surfaces of spinal fusion implant 5, e.g., substantially rectangular body 10, upper stabilizer 35 and/or lower stabilizer 40.

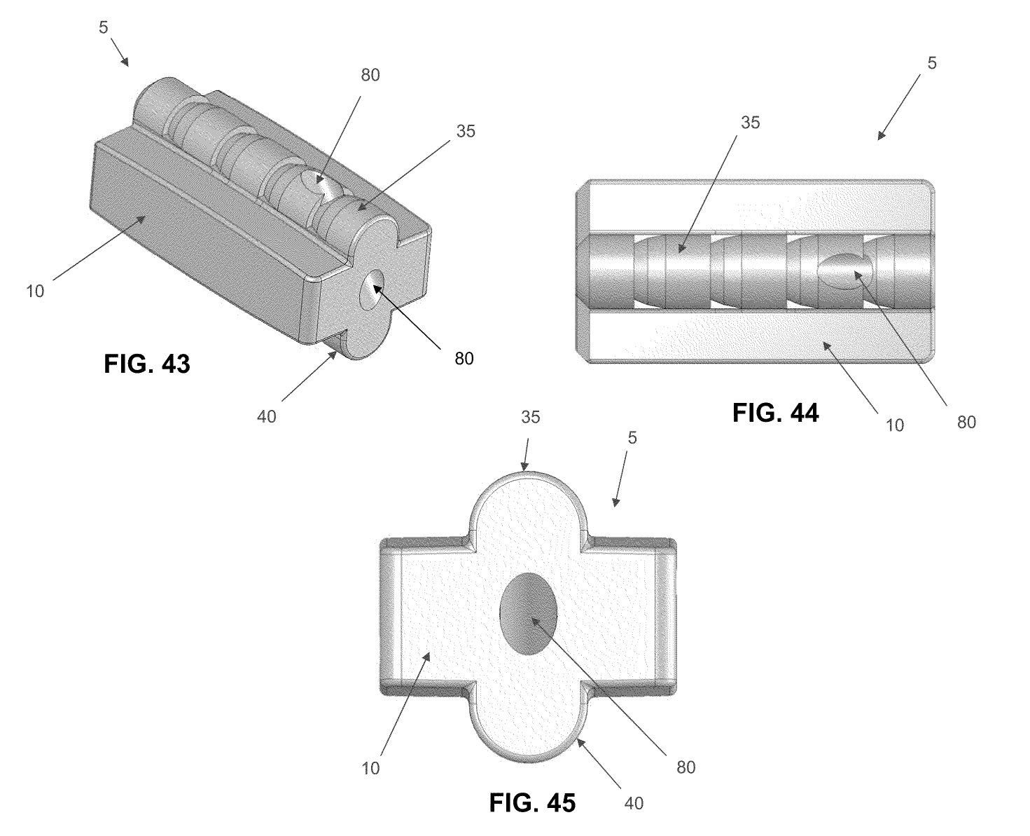

As seen in FIGS. 43-45, a crossbore 80 may also be provided in spinal fusion implant 5. Crossbore 80 may be used with a screw, K-Wire, suture, staple, pin or other fixation device so as to further secure spinal fusion implant 5 adjacent to a vertebral body or other structure. By way of example but not limitation, a screw may be used to screw spinal fusion implant 5 to an adjacent bony structure.

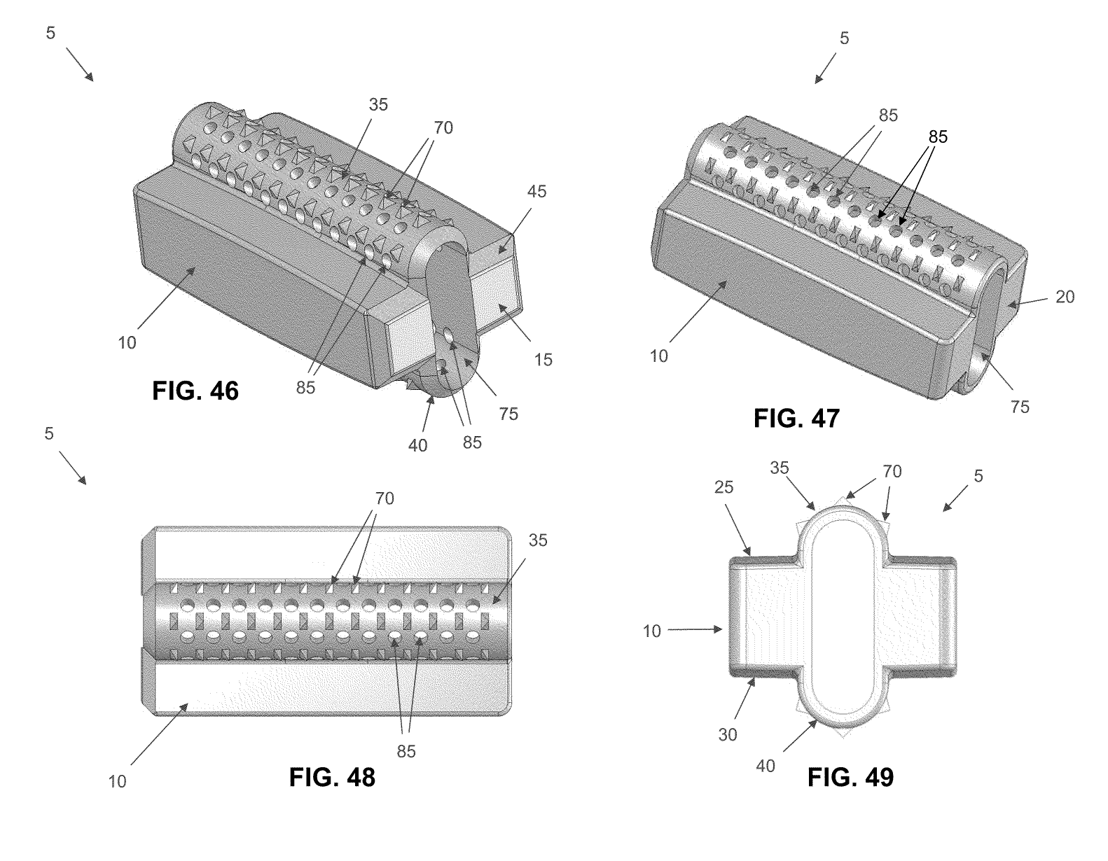

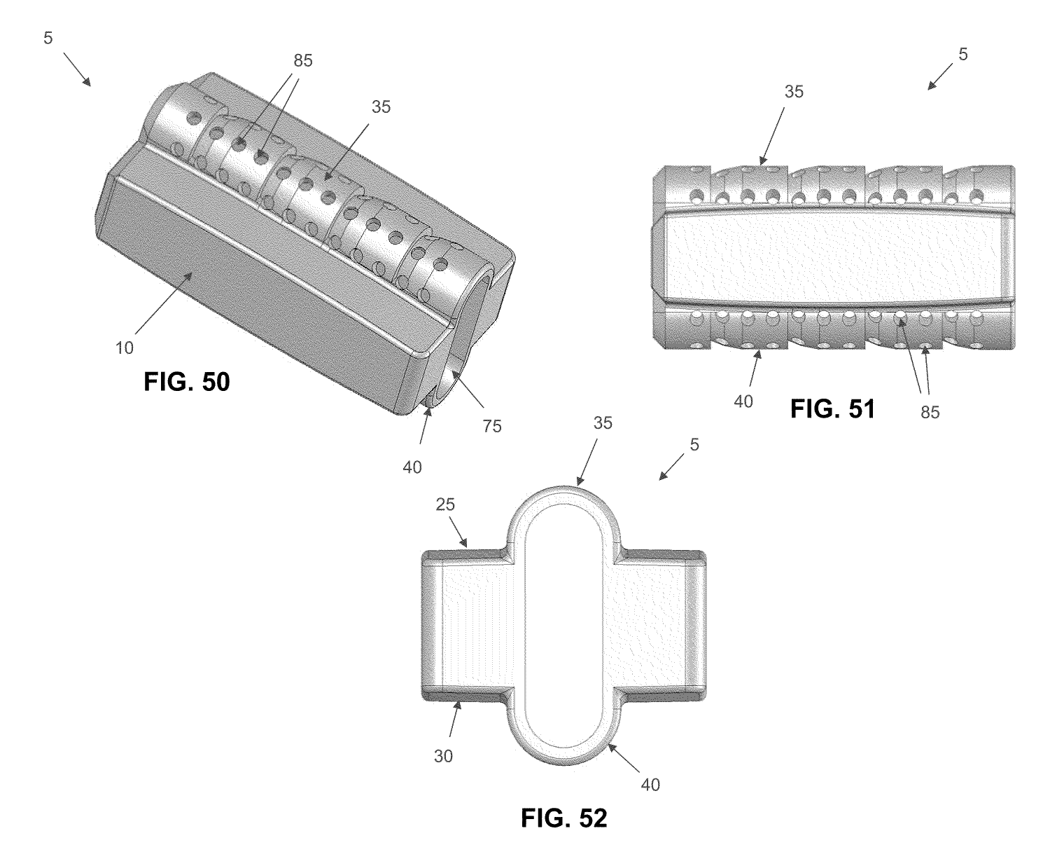

And as seen in FIGS. 46-49, and FIGS. 50-52, holes 85 may be provided in upper stabilizer 35 and lower stabilizer 40, with holes 85 communicating with longitudinally-extending opening 55.

Furthermore, if desired, one or the other of upper stabilizer 35 or lower stabilizer 40 may be omitted from spinal fusion implant 5.

### ADVANTAGES OF THE INVENTION

Numerous advantages are achieved by the present invention. Among other things, the present invention provides a fast, simple and easily reproduced approach for effecting spinal fusion. It also provides sufficient stabilization, where posterior plate or pedicle screws are not needed. And the present invention may be practiced using a minimally-invasive procedure or open surgical procedure.

### Applications to Joints Other than Intervetebral Joints

While spinal fusion implant 5 has been discussed above in the context of fusing an intervertebral joint, it should also be appreciated that the fusion implant may be used to stabilize and fuse any joint having anatomy similar to the intervertebral joint, i.e., a pair of opposing bony surfaces defining a gap therebetween, with the stabilizer of the fusion implant being sized to be positioned within the gap. By way of example but not limitation, the fusion implant may be used in small joints such as in the finger, toe, etc.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A spinal fusion implant comprising:
a substantially rectangular body having a distal end and a proximal end, and an upper surface and a lower surface; and
an upper stabilizer extending upwardly from the upper surface of the substantially rectangular body, and a lower stabilizer extending downwardly from the lower surface of the substantially rectangular body;
wherein (i) the distance between the distal end of the substantially rectangular body and the proximal end of the substantially rectangular body is long enough to span the distance between opposing cortical portions of a vertebral body and short enough to not extend substantially beyond the vertebral body, and (ii) the distance between the upper surface of the substantially rectangular body and the lower surface of the substantially rectangular body is substantially the same as the gap between opposing vertebral bodies in a spinal joint.

2. A spinal fusion implant according to claim 1 wherein the distal end of the substantially rectangular body is tapered.

3. A spinal fusion implant according to claim 1 wherein at least one of the upper stabilizer and the lower stabilizer has a substantially arcuate configuration.

4. A spinal fusion implant according to claim 1 wherein at least a portion of at least one of the upper stabilizer and the lower stabilizer has a frusto-conical configuration.

5. A spinal fusion implant according to claim 1 wherein at least one of the upper stabilizer and the lower stabilizer has a substantially rectangular configuration.

6. A spinal fusion implant according to claim 1 wherein the upper and lower stabilizers are aligned with one another.

7. A spinal fusion implant according to claim 1 wherein the spinal fusion implant is substantially solid.

8. A spinal fusion implant according to claim 1 wherein the spinal fusion implant includes at least one vertical opening therein to permit bone in-growth.

9. A spinal fusion implant according to claim 8 wherein the at least one vertical opening extends through the complete height of the spinal fusion implant.

10. A spinal fusion implant according to claim 8 wherein the at least one vertical opening comprises a blind hole.

11. A spinal fusion implant according to claim 8 wherein the at least one vertical opening is filled with a bone growth promoter.

12. A spinal fusion implant according to claim 1 wherein the substantially rectangular body includes at least one longitudinal opening therein.

13. A spinal fusion implant according to claim 1 further comprising at least one barb formed on the spinal fusion implant.

14. A spinal fusion implant according to claim 13 wherein the at least one barb is formed on the substantially rectangular body.

15. A spinal fusion implant according to claim 13 wherein the at least one barb is formed on at least one of the upper and lower stabilizers.

16. A spinal fusion implant according to claim 1 further comprising a fixation device for securing the spinal fusion implant in a joint.

17. A spinal fusion implant according to claim 16 wherein the fixation device comprises a screw.

18. A spinal fusion implant according to claim 1 wherein the upper surface of the substantially rectangular body and the lower surface of the substantially rectangular body are disposed substantially parallel to one another.

19. A spinal fusion implant according to claim 1 wherein the upper surface of the substantially rectangular body and the lower surface of the substantially rectangular body are disposed substantially non-parallel to one another.

20. A method for fusing a spinal joint, the method comprising the steps of:
providing a spinal fusion implant comprising:
a substantially rectangular body having a distal end and a proximal end, and an upper surface and a lower surface; and
an upper stabilizer extending upwardly from the upper surface of the substantially rectangular body, and a lower stabilizer extending downwardly from the lower surface of the substantially rectangular body;
wherein (i) the distance between the distal end of the substantially rectangular body and the proximal end of the substantially rectangular body is long enough to span the distance between opposing cortical portions of a vertebral body and short enough to not extend substantially beyond the vertebral body, and (ii) the distance between the upper surface of the substantially rectangular body and the lower surface of the substantially rectangular body is substantially the same as the gap between opposing vertebral bodies in a spinal joint;
deploying the spinal fusion implant in the spinal joint so that the substantially rectangular body is disposed between the opposing vertebral bodies of the spinal joint, with the upper stabilizer projecting into the upper vertebral body and the lower stabilizer projecting into the lower vertebral body; and
maintaining the spinal fusion implant in this position while fusion occurs.

21. A method according to claim 20 wherein the distal end of the substantially rectangular body is tapered.

22. A method according to claim 20 wherein at least one of the upper stabilizer and the lower stabilizer has a substantially arcuate configuration.

23. A method according to claim 20 wherein at least a portion of at least one of the upper stabilizer and the lower stabilizer has a frusto-conical configuration.

24. A method according to claim 20 wherein at least one of the upper stabilizer and the lower stabilizer has a substantially rectangular configuration.

25. A method according to claim 20 wherein the upper and lower stabilizers are aligned with one another.

26. A method according to claim 20 wherein the spinal fusion implant is substantially solid.

27. A method according to claim 20 wherein the spinal fusion implant includes at least one vertical opening therein to permit bone in-growth.

28. A method according to claim 27 wherein the at least one vertical opening extends through the complete height of the spinal fusion implant.

29. A method according to claim 27 wherein the at least one vertical opening comprises a blind hole.

30. A method according to claim 27 wherein the at least one vertical opening is filled with a bone growth promoter.

31. A method according to claim 20 wherein the substantially rectangular body includes at least one longitudinal opening therein.

32. A method according to claim 20 further comprising at least one barb formed on the spinal fusion implant.

33. A method according to claim 32 wherein the at least one barb is formed on the substantially rectangular body.

34. A method according to claim 32 wherein the at least one barb is formed on at least one of the upper and lower stabilizers.

35. A method according to claim 20 further comprising a fixation device for securing the spinal fusion implant in a joint.

36. A method according to claim 35 wherein the fixation device comprises a screw.

37. A method according to claim 20 wherein the upper surface of the substantially rectangular body and the lower surface of the substantially rectangular body are disposed substantially parallel to one another.

38. A method according to claim 20 wherein the upper surface of the substantially rectangular body and the lower surface of the substantially rectangular body are disposed substantially non-parallel to one another.

39. A fusion implant comprising:
a substantially rectangular body having a distal end and a proximal end, and a first surface and a second surface, the first surface and the second surface facing in substantially opposite directions; and
a first stabilizer extending away from the first surface of the substantially rectangular body, and a second stabilizer extending away from the second surface of the substantially rectangular body;
wherein (i) the distance between the distal end of the substantially rectangular body and the proximal end of the substantially rectangular body is long enough to span the distance between opposing cortical portions of a bone and short enough to not extend substantially beyond the bone, and (ii) the distance between the first surface of the substantially rectangular body and the second surface of the substantially rectangular body is substantially the same as the gap between opposing bones in a joint.

40. A method for fusing a joint, the method comprising the steps of:
providing a fusion implant comprising:
a substantially rectangular body having a distal end and a proximal end, and a first surface and a second surface, the first surface and the second surface facing in substantially opposite directions; and
a first stabilizer extending away from the first surface of the substantially rectangular body, and a second stabilizer extending away from the second surface of the substantially rectangular body;
wherein (i) the distance between the distal end of the substantially rectangular body and the proximal end of the substantially rectangular body is long enough to span the distance between opposing cortical portions of a bone and short enough to not extend substantially beyond the bone, and (ii) the distance between the first surface of the substantially rectangular body and the second surface of the substantially rectangular body is substantially the same as the gap between opposing bones in a joint;
deploying the fusion implant in the joint so that the substantially rectangular body is disposed between the opposing bones of the joint, with the upper stabilizer projecting into one bone of the joint and the lower stabilizer projecting into bone of the joint; and
maintaining the fusion implant in this position while fusion occurs.

41. A fusion implant comprising:
a body having a distal end and a proximal end, and a first surface and a second surface, the first surface and the second surface facing in different directions; and
at least one stabilizer extending away from the body;
wherein (i) the distance between the distal end of the body and the proximal end of the body is long enough to span the distance between opposing cortical portions of a bone and short enough to not extend substantially beyond the bone, and (ii) the distance between the first surface of the body and the second surface of the body is substantially the same as the gap between opposing bones in a joint.

42. A method for fusing a joint, the method comprising the steps of:
providing a fusion implant comprising:
a body having a distal end and a proximal end, and a first surface and a second surface, the first surface and the second surface facing in different directions; and
at least one stabilizer extending away from the body;
wherein (i) the distance between the distal end of the body and the proximal end of the body is long enough to span the distance between opposing cortical portions of a bone and short enough to not extend substantially beyond the bone, and (ii) the distance between the first surface of the body and the second surface of the body is substantially the same as the gap between opposing bones in a joint;
deploying the fusion implant in the joint so that the body is disposed between the opposing bones of the joint, with the at least one stabilizer projecting into one bone of the joint; and
maintaining the fusion implant in this position while fusion occurs.
